(19) United States
(12) Patent Application Publication
(10) Pub. No.: US 2008/0172114 A1
(43) Pub. Date: Jul. 17, 2008

(54) PHOTOTHERAPY HANDPIECE

(75) Inventors: Zafirios Gourgouliatos, Los Angeles, CA (US); David Chang, Encino, CA (US)

Correspondence Address: FOLEY & LARDNER LLP, 111 HUNTINGTON AVENUE, 26TH FLOOR, BOSTON, MA, 02199-7610, US

(73) Assignee: Lerner Medical Devices, Inc.

(21) Appl. No.: 12/014,462

(22) Filed: Jan. 15, 2008

Related U.S. Application Data
(60) Provisional application No. 60/880,887, filed on Jan. 17, 2007.
(60) Provisional application No. 60/880,883, filed on Jan. 17, 2007.
(60) Provisional application No. 60/880,812, filed on Jan. 17, 2007.
(60) Provisional application No. 60/880,813, filed on Jan. 17, 2007.

(51) Int. Cl. A61N 5/06

(52) U.S. Cl. 607/94

(57) ABSTRACT

A phototherapy device is disclosed including: a handpiece; and a beamshaping element housed in the handpiece. The beamshaping element is adapted to receive a beam of light from an ultraviolet light source, modify the shape of the beam, and direct the modified beam to provide illumination of a region of a treatment surface, the region, having a cross section at the treatment surface with an irregularly shaped boundary. In some embodiments, the irregularly shaped boundary is composed of one or more segments, each segment being sinusoid-like. In some embodiments, the beamshaping element includes a screen disposed between the light source and the treatment surface, the screen including a light transmissive region with an irregularly shaped boundary, and where the irregularly shaped boundary of the illuminated area corresponds to the irregularly shaped boundary of the light transmissive region.

### CROSS REFERENCE TO RELATED APPLICATIONS

The present application claims priority from U.S. Provision Application Ser. No. 60/880,883, U.S. Provisional Application Ser. No. 60/880,8127 U.S. Provisional Application Ser. No. 60/880,813, U.S. Provisional Application Ser. No. 60/880,887, each filed Jan. 17, 2007, and each of which is incorporated by reference herein in its entirety.

### BACKGROUND

This disclosure relates to treatments for inflammatory diseases of the skin, and more specifically to methods for devices and treating ultraviolet light-sensitive dermatoses.

When the disease is localized, targeted devices can direct the light over and around the affected area. A side-effect of this treatment is hyperpigmentation (tanning) of the exposed skin. Typical such devices produce square or round footprints of light on the skin with clearly defined edges. The hyperpigmentation patterns are produced with the exact shape of the footprint of the light that exposed the skin. A typical exposure is in the order of 1 to 30 seconds. The onset of hyperpigmentation is typically within 2 to 12 hours of light exposure. This delayed onset does not give the operator of the device any immediate specific marker to indicate where the skin was exposed. When the lesion to be treated is larger than the footprint of the light pattern, light is deposited on the skin in a tile fashion, one such exposed area next to the previous one. As these exposures are laid on the skin, due to luck of an indication and limitation of accuracy in the placement, the exposed areas either overlap or leave some spaces or gaps of unexposed skin. This results in hyperpigmented areas due to a single exposure or double exposure interlaced with non-tanned areas. These patterns do not look natural and are unsightly. As mentioned above, the onset of the hyperpigmentation is within the day of exposure to light. This condition lasts from a few days to sometimes 2-3 months, depending on the intensity of the exposure and the skin type of the patient.

To overcome this problems operators place dots over the skin with an ink marker to guide the placement or attach a small rubber stamp with ink to the handpiece that leaves a mark every time an area is exposed. Both of them do not provide a satisfactory solution.

### SUMMARY

The present disclosure describes therapy devices and methods for effective treatment of inflammatory dermatoses such as psoriasis. The devices includes an optical source including means for generating ultraviolet (UV) light (radiation) in a predetermined spectral range, and a light delivery apparatus that produces a pattern of light on the skin surface that has sinusoid-like edges, e.g., perforated “postal stamp” type edges comprising of small semicircle indentations and extensions.

The inventors have found a solution to the above described problem with the use of patterns that have edges comprised of sinusoid-like lines. These patterns, when tiled next to each other, if they are not perfectly aligned, produce hyperpigmentation patterns that have curved edges and the hyperpigmented areas from the single exposure or double exposure as well as the non-tanned areas resemble pattern that naturally occur on human skin. These light induced patterns can be oval, round, or have the forms of curved lines. On the contrary, hyper pigmentation and non pigmented areas resulting from exposures produced by prior devices that have straight edges (straight lines in the sense when a transparent flat piece of glass is pressed on the skin so that the area below it becomes flat).

In addition, patterns that have perforated sinusoid-like “postal stamp” type edges, comprising of, for example, small semicircle indentations and extensions, produce more natural looking patterns in the double exposed, exposed or unexposed areas of the skin.

When such devices are combined with photosensitizers, the result is similar and quite often more pronounced.

In one aspect, a phototherapy device is disclosed including: a handpiece; and a beamshaping element housed in the handpiece. The beamshaping element is adapted to receive a beam of light from an ultraviolet light source, modify the shape of the beam, and direct the modified beam to provide illumination of a region of a treatment surface, the region having a cross section at the treatment surface with an irregularly shaped boundary. In some embodiments, the irregularly shaped boundary is composed of one or more segments, each segment being sinusoid-like. In some embodiments, the beamshaping element includes a screen disposed between the light source and the treatment surface, the screen including a light transmissive region with an irregularly shaped boundary, and where the irregularly shaped boundary of the illuminated area corresponds to the irregularly shaped boundary of the light transmissive region.

In some embodiments, the irregularly shaped boundary is described by one of the group of: a square with slanted or curved edges, a triangle with slanted or curved edges, square with postage stamp type edges, a triangle with postage stamp type edges, a polygon with one or more slanted, curved, or postage stamp type edges.

Some embodiments include a light guide configured to deliver light from the ultraviolet source to the handpiece.

Some embodiments include the ultraviolet light source. The light source may be housed in the handpiece. In some embodiments, the light source includes at least one from the group of: a light emitting diode, a lamp, a laser, an excimer laser, a diode laser, an excimer gas discharge lamp. In some embodiments, the light source includes: a first source of light at a first wavelength; and a wavelength converter adapted to convert at least a portion of the light from the first source to provide a beam of ultraviolet light.

In some embodiments, the light source has a spectral output within the range of 280 nm to 320 nm, within the range of 308 nm to 320 nm, or within the range of 320 nm to 380 nm.

Some embodiments include a control module in communication with the beam shaping element or light source, the control module adapted to selectively adjust the duration or intensity of the illumination.

Some embodiments include a distance gauge extending between an end proximal to the handpiece and an end distal the handpiece. Some such embodiments include a sensor adapted to sense and indicate the contact of the distal end of the distance gauge to the treatment surface. In some embodiments, the distance gauge is detachably affixed to the handpiece. In some embodiments, the distance gauge is autoclavable. In some embodiments, the control module is adapted to inhibit the illumination of the treatment surface when the sensor does not indicate contact of the distal end of the distance gauge to the treatment surface.

Some embodiments include a power supply adapted to supply power to the ultraviolet light source. The power supply may be housed in the handpiece.

In some embodiments, the beam shaping element includes one or more lenses. The one or more lenses may be adapted to direct light transmitted through the screen to the treatment area.

Some embodiments include a support including the aforementioned screen, where the support is retractably housed in the handpiece.

In another aspect, a treatment system is disclosed including a handpiece including a beamshaping element, the beamshaping element adapted to receive a beam of light from an ultraviolet light source, modify the shape of the beam; and direct the modified beam to provide illumination of a region of a treatment surface, the region having a cross section at the treatment surface with an irregularly shaped boundary; and a plurality of screens adapted to be interchangeably received by the beam shaping element at a position disposed between the light source and the treatment surface, each of the screens including a light transmissive region with an irregularly shaped boundary, where the irregularly shaped boundary of the illuminated area corresponds to the irregularly shaped boundary of the light transmissive region. The irregularly shaped boundary is composed of one or more segments, each segment being sinusoid-like. The shape of the boundary of the light transmissive region of each screen varies from screen to screen.

Some embodiments include a support including retractably housed in the handpiece, the support including one or more of the plurality of screens.

In yet another aspect, a method of treating i skin disease is disclosed including:

A. illuminating a region of skin within a treatment area to provide an effective dose of treatment light, the area having a an irregularly shaped boundary; and

B. successively repeating step A to tile substantially all of the treatment area with illuminated regions, each of the regions having a cross section at the treatment surface with a an irregularly shaped boundary.

In some embodiments, the irregularly shaped boundary is composed of one or more segments, each segment being sinusoid-like. In some embodiments, the irregularly shaped boundary of at least one of the regions is described by one of the group of: a square with slanted or curved edges, a triangle with slanted or curved edges, a square with postage stamp type edges, a triangle with postage stamp type edges, a polygon with one or more slanted, curved, or postage stamp type edges.

In some embodiments, the effective dose is about one minimum erythema dose or more.

In some embodiments, the illuminating a region of skin includes providing illumination from a treatment device, the treatment device of any of the types described above.

It is to be understood that, as used herein, skin disease includes inflammatory skin disease such as psorasis, vitiligo, pigmentation loss, and other disorders.

Various embodiments may include any of the above described features, alone or in any combination. These and other features will be more fully appreciated with reference to the following detailed description which is to be read in conjunction with the attached drawings.

### DESCRIPTION OF THE DRAWINGS

The foregoing and other objects of this disclosure, the various features thereof, may be more fully understood from the following description, when read together with the accompanying drawings in which:

FIG. 1 is a perspective view showing an exemplary phototherapy handpiece;

FIG. 2 is a perspective view with the enclosure cover removed, showing the parts of the exemplary phototherapy device;

FIG. 3 shows two exemplary patterns produced by the device;

FIG. 4 shows two exemplary patterns with perforated edges (show in detail in insets) produced by the device;

FIG. 5 is a photograph showing an exemplary phototherapy device in clinical use; and

FIG. 6 is a photograph showing a treatment area tiled with regions of treatment illumination having cross sections at the treatment surface with irregularly shaped boundaries (shown in detain in inset).

### DESCRIPTION

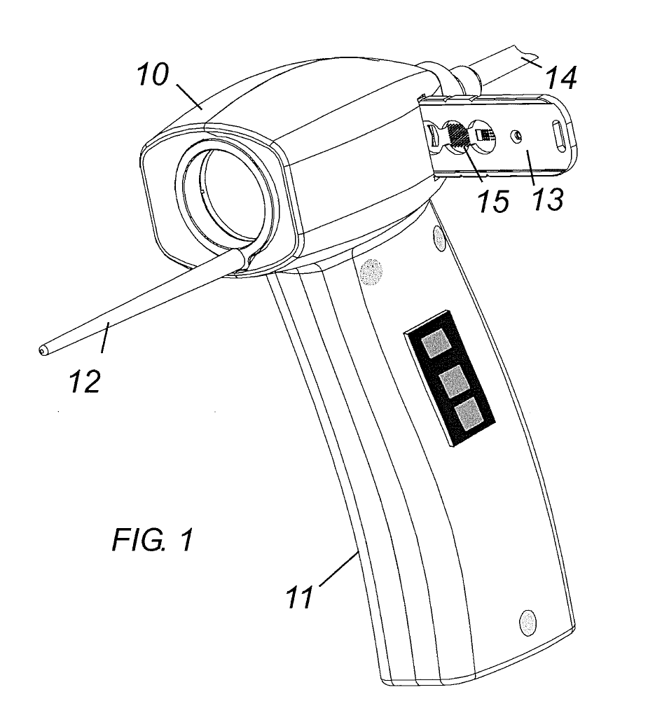

Therapeutic device 10 is shown in FIG. 1, and includes a light delivery apparatus including body member 11 with a handle for convenient use, a distance gage 12 and a plurality of light transporting and shaping elements such as lenses and a light transporting waveguide 14 detachably affixed therefrom. The light goes through one selected screen 15 affixed on a support 13 and the light, having the pattern of the screen is projected onto the skin with the light transporting and shaping elements.

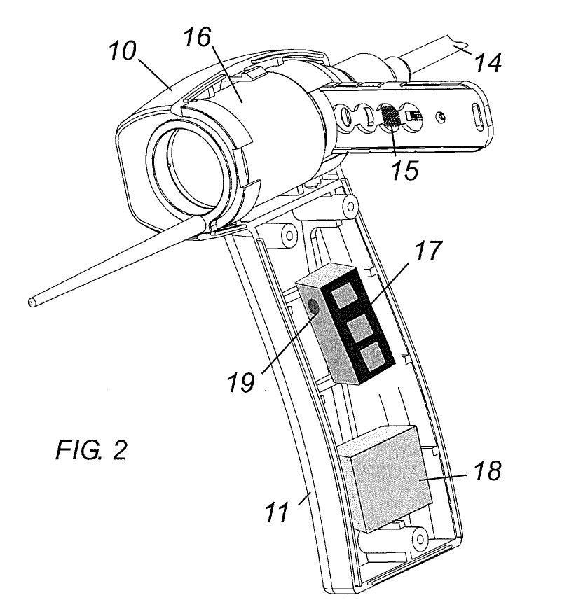

As shown in detail in FIG. 2, body member 11 contains the light transporting and shaping elements 16 that can made out of a single lens or an assembly of lenses affixed in proximity to the a light transporting waveguide 14 and screen 15. Preferably, body member 11 has substantially no UV-transferring abilities, and is formed of a molded resinous material, such as plastic, rubber, and the like.

Body member 11 can include controls such as triggers for light activation as well as display and input elements 17, an auditory signal transducer 19 for operator warnings, etc. In addition it can have a cord that wraps around the wrist of the operator to prevent it from falling to the ground accidentally.

In some embodiments, the power supply and control module 18 may instead be inside the body member. The electrical power source can also be embodied in the body member in the form of batteries, preferably rechargeable. The activation of the device can be external with a switch, attached to the control module, or connected with a cable, or in the form of a foot pedal or with wireless communication.

The distance gage can be combined with a sensor to detect contact between the tip of the gage and the skin of the patient. This will provide additional safety by preventing the device to be activated unless the tip is in contact with the skin.

The light power can be provided by, but not limited to, laser, excimer laser, excimer gas discharge lamps, Light Emitting Diode (LED), or lamp with high pressure, short arc or other type. The light producing element can be inside or external to the body member 11 with the control module connected with electrical cable to the body member or embodied 18 in the body member enclosure as mentioned above.

In some embodiments, the optical power of a different wavelength can be produced inside or outside the enclosure and converted to light in the UV-B spectrum by a wavelength converter consisting of photon excitable material. In the case that the light is produced externally to the handpiece, it will be transported to the handpiece with a light guide.

If the light of the embodiment described in the last paragraph is produced outside the handpiece, the light power will be transported to the hand piece vial a flexible lightguide or fiber optic. The flexible lightguide can be of the type of liquid filled pipe. The fiber optic can be of quartz or fused silica material with glass or polymer cladding.

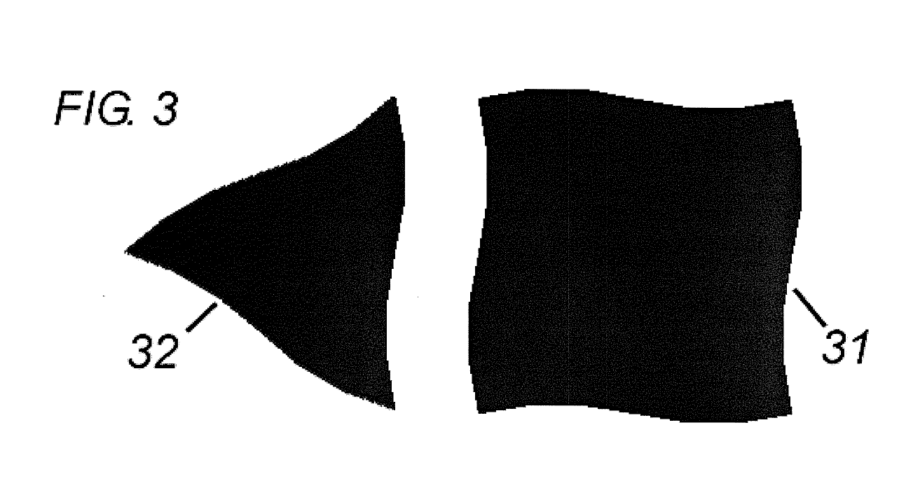

Light pattern producing screens can be retractable and interchangeable. One such embodiment is shown in FIG. 3.

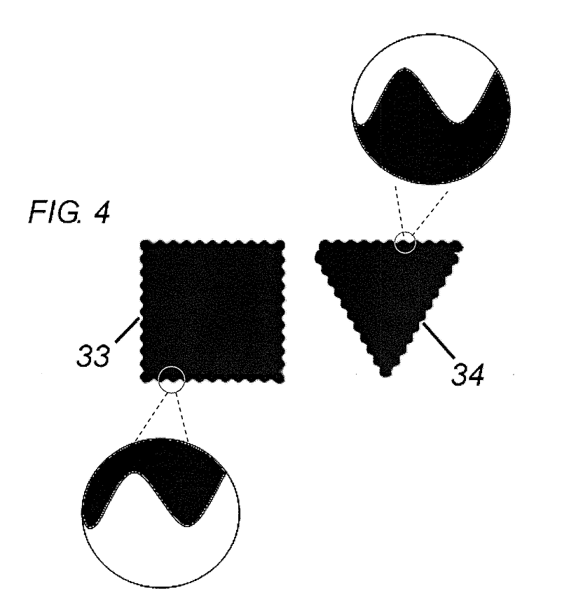

Various patterns, i.e. cross sections of the illuminated region at the treatment surface, are shown in FIGS. 3 and 4. The patterns have irregularly shaped boundaries, for example a square with the slanted edges 31, triangle with similar edges 32 and square 33 or triangle 34 with perforated, “postal stamp” type edges (see detailed insets). Differing patters will give the best results, depending on the area of the skin that is treated. In each case the irregularly shaped boundary of the patterns is made up of sinusoid-like segments. By sinusoid-like, it is meant that these segments resemble a sinusiod, i.e. they are composed of multiple, connected arcs of alternating concavity/convexity.

The distance gage is detachably attached to body member, made of hardy material, either for single use or autoclavable or able to be gas sterilized.

The advantage of this device is that localized areas of the skin may be treated without exposing the entire body to photosensitizers and/or to UV light.

In a preferred embodiment, the light producing elements can emit light in the spectral range of 280-320 nm, preferably in the 308-320 nm for dermatoses that respond to UV-B light or Narrow band UV-B light. Alternatively they can emit light in the 320-380 nm for dermatoses that respond to UV-A light, as well as in other spectral bands of visible or infrared light for dermatoses or skin conditions that respond to specific wavelengths.

Preferred embodiments of the method of treating an inflammatory dermatosis using the aforementioned device are as follows.

For UV-B phototherapeutic treatment, exposure with prescribed dose will be tiled on the skin until affected area is treated. Depending on the treatment protocol, exposure can start with approximately one minimum erythema dose (MED), or a multiple of it. Subsequent treatment times would increase if needed and as tolerated by the patient.

In practice, the delivered exposure dose (fluence) needs to be controlled to within about 40% absolute. Both short-term and long-term output stability, including solarization of optical elements, are considerations affecting dosimetry. If the source output is stable (e.g., less than 10% variation of UV irradiance) after a short warm-up period, over the duration of one treatment (typically a few seconds per exposure), then a timer type of device to control delivered dose based on a measured irradiance is appropriate. If the output is unstable, an integrating dosimeter is required. The ideal system would be stable, might require the user to point the output onto a detector which measures irradiance appropriately, and then enter the desired dose in J/cm.sup.2.

Of course, certain areas of the skin such as the nails could also be treated as described above.

### CLINICAL EXAMPLES

The following describes the clinical use of a phototherapy device and methods of the type described above.

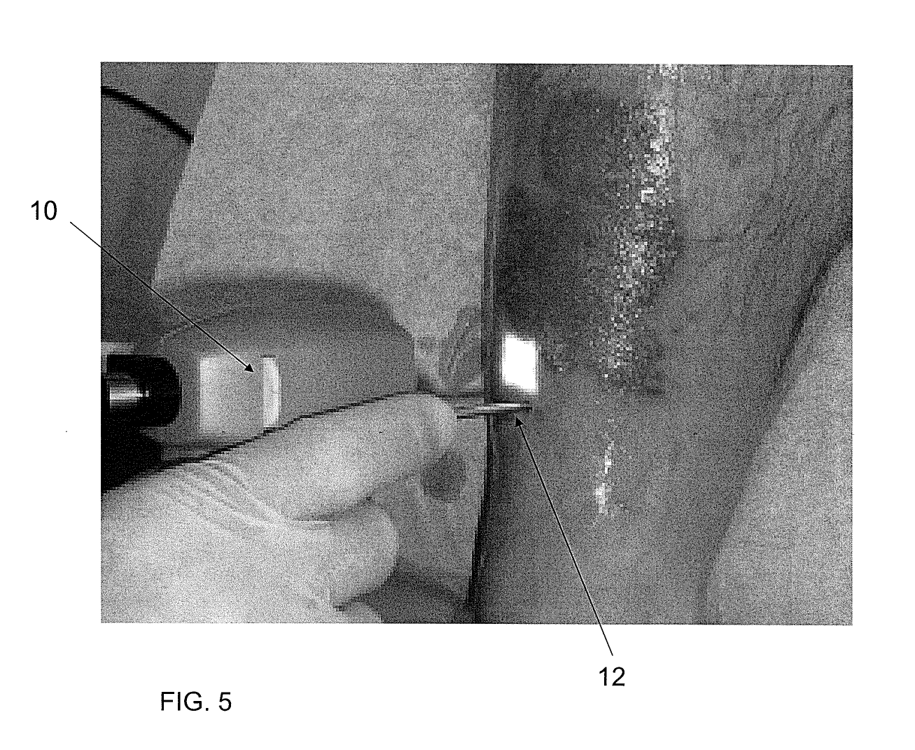

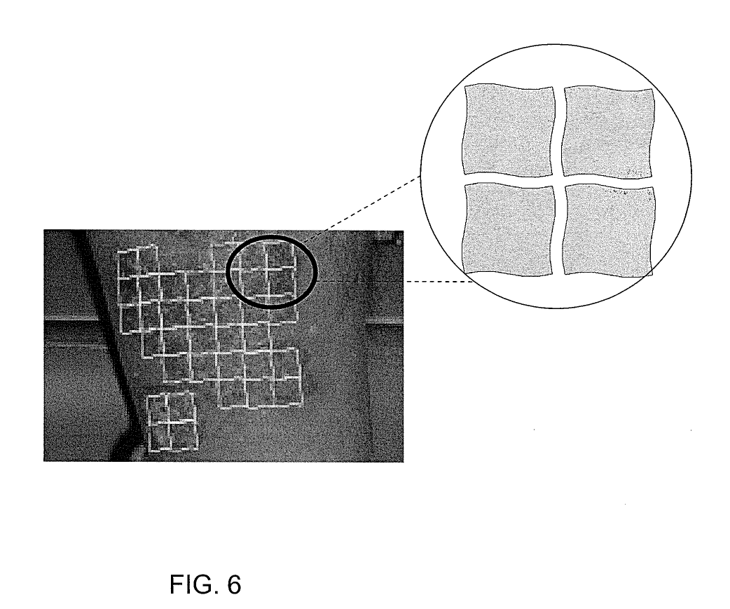

As shown in FIG. 5, a handpiece 10 of the type described above was used. The handpiece had inserts with patterns of various shapes and sizes to match the lesions. Lesions having size larger than the spot illuminated by the handpiece were treated with the tiling method described above. This method involved laying small areas of illumination next to each other until the whole affected area was exposed. As shown in FIG. 6, for tiling, the handpiece illuminated areas having a cross section at the treatment surface with an irregular boundary composed of sinusoid-like segments. Four such areas are shown in detain in the inset. Note that although the boundaries of the areas shown in the inset are spaced apart, in general they may abut or overlap. As noted above, an advantage of this feature was that when the tiling was not in perfect alignment, the resulting hyperpigmentation patterns (tanning) on healthy skin due to double exposure or unexposed spaces would have curved edges and look like natural occurring pigmentation. This feature is particularly useful not only in psoriasis but also in eczema and vitiligo treatments.

Exemplary treatment dose levels rare shown in FIG. 7 for various skin types as will be recognized by those skilled in the art. In the examples below the initial dose of 2 MEDs was increased by approximately 20% per treatment. The device produced 100 mW/cm² at full output. For the Type II and Type III patients of this study, the exposure time and intensity varied from 1 to 8 seconds per spot to deliver the appropriate dose. If the patient complained of burning sensation discomfort, the dose was not increased or the increase was moderated. The same applied on the few occasions the patient skipped a treatment

As described in detail in Rubenstein et. al. Journal of the American Academy of Dermatology Volume 56, Issue 2, Supplement 2, February 2007, Page AB167, multiple patients treated using the device and techniques described above experienced reduction or elimination of psoriasis treatments as a result of treatments of the type described above.

One or more or any part thereof of the control, sensing, or other techniques described above can be implemented in computer hardware or software, or a combination of both. The methods can be implemented in computer programs using standard programming techniques following the method and figures described herein. Program code is applied to input data to perform the functions described herein and generate output information. The output information is applied to one or more output devices such as a display monitor. Each program may be implemented in a high level procedural or object oriented programming language to communicate with a computer system. However, the programs can be implemented in assembly or machine language, if desired. In any case, the language can be a compiled or interpreted language. Moreover, the program can run on dedicated integrated circuits preprogrammed for that purpose.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during program execution. The technique can also be implemented as a computer-readable storage medium, configured with a computer program, where the storage medium so configured causes a computer to operate in a specific and predefined manner to perform the functions described herein.

As used herein the terms “light,” “optics,” “optical,” etc are to be understood to include electromagnetic radiation both within and outside of the visible spectrum, including, for example, ultraviolet radiation.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A phototherapy device comprising.
a handpiece; and
a beamshaping element housed in the handpiece;
wherein the beamshaping element is adapted to
receive a beam of light from an ultraviolet light source,
modify the shape of the beam, and
direct the modified beam to provide illumination of a region of a treatment surface, said region having a cross section at the treatment surface with an irregularly shaped boundary.

2. The apparatus of claim 1, wherein the irregularly shaped boundary is composed of one or more segments, each segment being sinusoid-like.

3. The apparatus of claim 2, wherein the beamshaping element comprises a screen disposed between the light source and the treatment surface, said screen comprising a light transmissive region with an irregularly shaped boundary, and wherein the irregularly shaped boundary of the illuminated area corresponds to the irregularly shaped boundary of the light transmissive region.

4. The apparatus of claim 3, farther comprising a light guide configured to deliver light from the ultraviolet source to the handpiece.

5. The apparatus of claim 4, further comprising the ultraviolet light source.

6. The apparatus of claim 5, wherein the light source is housed in the handpiece.

7. The apparatus of claim 3, wherein the light source comprises at least one from the group of, a light emitting diode, a lamps a laser, an excimer laser, a diode laser, an excimer gas discharge lamp.

8. The apparatus of claim 5, wherein the ultraviolet light source comprises:
a first source of light at a first wavelength; and
a wavelength converter adapted to convert at least a portion of the light from the first source to provide a beam of ultraviolet light.

9. The apparatus of claim 5, wherein the light source has a spectral output within the range of 280 nm to 320 nm.

10. The apparatus of claim 8, wherein the light source has a spectral output within the range of 308 nm to 320 nm.

11. The apparatus of claim 4, wherein the light source has a spectral output within the range of 320 nm to 380 nm.

12. The apparatus of claim 3, wherein the irregularly shaped boundary is described by one of the group of: a square with slanted or curved edges, a triangle with slanted or curved edges, a square with postage stamp type edges, a triangle with postage stamp type edges, a polygon with one or more slanted, curved, or postage stamp type edges.

13. The apparatus of claim 3, further comprising a control module in communication with the beam shaping element or light source, said control module adapted to selectively adjust the duration or intensity of the illumination.

14. The apparatus of claim 13, further comprising:
a distance gauge extending between an end proximal to the handpiece and an end distal the handpiece.

15. The apparatus of claim 14, comprising a sensor adapted to sense and indicate the contact of the distal end of the distance gauge to the treatment surface.

16. The apparatus of claim 14, wherein the distance gauge is detachably affixed to the handpiece.

17. The apparatus of claim 14, wherein the distance gauge is autoclavable.

18. The apparatus of claim 15, wherein the control module is adapted to inhibit the illumination of the treatment surface when the sensor does not indicate contact of the distal end of the distance gauge to the treatment surface.

19. The apparatus of claim 3, further comprising a power supply adapted to supply power to the ultraviolet light source.

20. The apparatus of claim 19, wherein the power supply is housed in the handpiece.

21. The apparatus of claim 3, wherein the beam shaping element comprises one or more lenses.

22. The apparatus of claim 21, wherein the one or more lenses are adapted to direct light transmitted through the screen to the treatment area.

23. The apparatus of claim 3, further comprising a support comprising the screen, wherein the support is retractably housed in the handpiece.

24. A treatment system comprising
a handpiece comprising a beamshaping element, said beamshaping element adapted to receive a beam of light from an ultraviolet light source, modify the shape of the beam; and direct the modified beam to provide illumination of a region of a treatment surface, said region having a cross section at the treatment surface with an irregularly shaped boundary; and
a plurality of screens adapted to be interchangeably received by the beam shaping element at a position disposed between the light source and the treatment surface, each of said screens comprising a light transmissive region with an irregularly shaped boundary,
wherein the irregularly shaped boundary of the illuminated area corresponds to the irregularly shaped boundary of the light transmissive region;
wherein the irregularly shaped boundary is composed of one or more segments, each segment being sinusoid-like; and
wherein the shape of the boundary of the light transmissive region of each screen varies from screen to screen.

25. The system of claim 24, further comprising a support comprising retractably housed in the handpiece, said support comprising one or more of the plurality of screens.

26. A method of treating inflammatory skin disease comprising:
A. illuminating a region of skin within a treatment area to provide an effective dose of treatment light, said area having a an irregularly shaped boundary; and
B. successively repeating step A to tile substantially all of the treatment area with illuminated regions, each of said regions having a cross section at the treatment surface with a an irregularly shaped boundary.

27. The method of claim 26, wherein the irregularly shaped boundary is composed of one or more segments, each segment being sinusoid-like;

28. The method of claim 27, wherein the effective dose is about one minimum erythema dose or more.

29. The method of claim 27, wherein the illuminating a region of skin within a treatment area to provide an effective dose of treatment light, said area having an irregularly shaped boundary comprises providing illumination from a treatment device, said treatment device comprising:
a handpiece; and
a beamshaping element housed in the handpiece;
wherein the beamshaping element is adapted to receive a beam of light from an ultraviolet light source, modify the shape of the beam, and direct the modified beam to provide illumination of a region of a treatment surface, said region having a cross section at the treatment surface with an irregularly shaped boundary,
wherein the beamshaping element comprises a screen disposed between the light source and the treatment surface, said screen comprising a light transmissive region with an irregularly shaped boundary, and wherein the irregularly shaped boundary of the illuminated area corresponds to the irregularly shaped boundary of the light transmissive region.

30. The method of claim 25, wherein the irregularly shaped boundary of at least one of the regions is described by one of the group of: a square with slanted or curved edges, a triangle with slanted or curved edges, a square with postage stamp type edges, a triangle with postage stamp type edges, a polygon with one or more slanted, curved, or postage stamp type edges.
